(19) United States
(12) Patent Application Publication
(10) Pub. No.: US 2017/0258690 A1
(43) Pub. Date: Sep. 14, 2017

(54) COMPOSITION COMPRISING AN AQUEOUS PHASE AND A FATTY PHASE THAT ARE VISUALLY DISTINCT USED FOR HAIR TREATMENT

(71) Applicant: L'OREAL, Paris (FR)

(72) Inventors: Anne-sophie BRAC DE LA PERRIERE, Paris (FR); Valérie VIEIRA, Pomponne (FR); Ségolène DE MENTHIERE, Saintouen (FR)

(73) Assignee: L'OREAL, Paris (FR)

(21) Appl. No.: 15/529,293

(22) PCT Filed: Nov. 27, 2015
(86) PCT No.: PCT/EP2015/077861

§ 371 Date: May 24, 2017

(30) Foreign Application Priority Data
Nov. 27, 2014 (FR) 1461598

(51) Int. Cl. A61K 8/03; A61K 8/31; A61K 8/86; A61K 8/49; A61K 8/34; A61K 8/41; A61Q 5/06; A61K 8/19

(52) U.S. Cl. CPC A61K 8/03; A61Q 5/06; A61K 8/31; A61K 8/19; A61K 8/498; A61K 8/342; A61K 8/41; A61K 8/86; A61K 2800/48

(57) ABSTRACT

The present invention relates to a cosmetic hair treatment process comprising the step of applying on the hair a composition comprising:—a fatty phase comprising: a) at least one oil; b) at least one fatty-phase thickener; c) at least one water-insoluble mineral particulate compound, other than the fatty-phase thickener b);—an aqueous phase comprising at least one aqueous-phase thickening polymer, the two phases being visually distinct.

The present invention relates to a composition comprising a fatty phase and an aqueous phase that are visually distinct, the aqueous phase comprising at least one aqueous-phase thickener and the fatty phase comprising at least one oil, one fatty-phase thickener and one water-insoluble mineral particulate compound used in a hair treatment process.

In the field of styling, in particular among hair products intended for shaping and/or holding the hairstyle, hair compositions are generally in the form of hair gels, lotions, mousses or sprays.

In particular, hair gels allow good hold of the head of hair. However, many hair gels do not give the hair sufficient cosmeticity and the final result lacks a natural look.

In addition, the majority of hair gels have a uniform appearance that is not very appealing.

It has already been proposed to make compositions comprising visually distinct phases, as described, for example, in patent applications WO 2006/093 742, WO 2006/042 179, WO 2006/010 090 or WO 2007/004 200. In practice, the weight ratio between the various phases can only generally vary within a limited range of values and the preparation of these compositions is often difficult, for a stability of the compositions obtained that is not entirely satisfactory.

Haircare products often use conditioning agents, especially oils. Anhydrous compositions containing oils very often lead to hair that feels too greasy. In aqueous compositions, a limitation is very often posed by the very low solubility of oils in water, generally imposing the use of surfactants in aqueous compositions containing these oils, often with, as a corollary, an opaque final appearance of creamy emulsion type.

There is thus a real need to find cosmetic compositions, which afford haircare and which allow easy formulation of oils in aqueous medium.

There is also a need to find styling compositions which make it possible to give volume to the hairstyle and allow good hairstyle hold while at the same time giving the hair cosmeticity.

There is also a need to find cosmetic compositions, especially for styling, which make it possible to obtain a hairstyle with a natural look.

There is also a need to find cosmetic compositions that have a novel, more attractive aesthetic look.

The Applicant has discovered that the choice of an aqueous phase comprising at least one aqueous-phase thickener and of a fatty phase comprising at least one oil, at least one fatty-phase thickener and at least one water-insoluble mineral particulate compound, in which the two phases are visually distinct, makes it possible to satisfy at least one of these needs.

Thus, one subject of the present invention is a cosmetic hair treatment process comprising the step of applying on the hair a composition comprising:
a fatty phase comprising:
   a) at least one oil;
   b) at least one fatty-phase thickener;
   c) at least one water-insoluble mineral particulate compound, other than the fatty-phase thickener b);
an aqueous phase comprising at least one aqueous-phase thickening polymer, the two phases being visually distinct.

The present invention makes it possible to prepare aesthetic compositions that care for and style the hair for instance that give volume, smooth the hair, define curls or give a natural and flexible shape to the hairstyle.

It is thus possible to obtain a composition which comprises a relatively large amount of oil.

A subject of the invention is also the use of the said composition for caring for the hair and/or for shaping the hair.

In the text hereinbelow, the expression “at least one” is equivalent to “one or more” and, unless otherwise indicated, the limits of a range of values are included in that range.

The composition according to the invention comprises two visually distinct phases. The term “two visually distinct phases” means that the phases may be distinguished from each other by a person's naked eye, unlike phases forming emulsions or dispersions of homogeneous particles. Preferably, at least one of the phases occupies zones forming volutes or marbling, preferably more than 1 cm in length. Preferably, one of the phases is not in the form of globules. More preferably, none of the phases is in the form of globules.

The two phases are visually distinct in a stable manner, i.e. the zones occupied by the two phases do not move in response to a simple converting of the container containing them, without any other stress applied to the composition. The two phases are incapable of mixing together when the container containing them is shaken. The two phases especially do not constitute liquid double-phases for which two distinct phases occupy zones one above the other and which, when the container is inverted, mix together.

As mentioned previously, the composition according to the invention comprises a fatty phase.

The fatty phase of the composition in accordance with the invention comprises at least one oil.

The term “oil” means any fatty substance that is in liquid form at room temperature (25° C.) and at atmospheric pressure.

The oil(s) present in the composition may be volatile or non-volatile.

The volatile or non-volatile oils may be hydrocarbon-based oils, in particular of animal or plant origin, synthetic oils, silicone oils or fluoro oils, or mixtures thereof.

For the purposes of the present invention, the term “silicone oil” means an oil comprising at least one silicon atom, and in particular at least one Si-O group.

The term “hydrocarbon-based oil” means an oil mainly containing hydrogen and carbon atoms, and optionally oxygen, nitrogen, sulfur and/or phosphorus atoms. A hydrocarbon-based oil does not comprise any silicon atoms.

### Non-volatile Oils

For the purposes of the present invention, the term “non-volatile oil” means an oil having a vapour pressure of less than 0.13 Pa (0.01 mmHg).

The non-volatile oils may be chosen in particular from non-volatile hydrocarbon-based oils, which may be fluorinated, and/or non-volatile silicone oils.

As non-volatile hydrocarbon-based oils that are suitable for use in the invention, mention may be made in particular of:
hydrocarbon-based oils of animal origin;
hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C4 to C24, these chains possibly being linear or branched, and saturated or unsaturated; these oils are especially heptanoic or octanoic triglycerides, sweet almond oil, argan oil, avocado oil, groundnut oil, camellia oil, safflower oil, beauty-leaf oil, rapeseed oil, copra oil, coriander oil, marrow oil, wheatgerm oil, jojoba oil or liquid jojoba wax, linseed oil, macadamia oil, corn germ oil, hazelnut oil, walnut oil, vernonia oil, apricot kernel oil, olive oil, evening primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil; or alternatively caprylic/capric acid triglycerides, for instance those sold by the company Stearineries Dubois or those sold under the names Miglyol 810®, 812® and 818® by the company Dynamit Nobel, and the refined plant perhydrosqualene sold under the name Fitoderm by the company Cognis;
hydrocarbon-based oils of mineral or synthetic origin, for instance:
   synthetic ethers containing from 10 to 40 carbon atoms;
   linear or branched hydrocarbons of mineral or synthetic origin, such as petroleum jelly, polydecenes, hydrogenated polyisobutene such as Parleam, and squalane, and mixtures thereof, and in particular hydrogenated polyisobutene;
   synthetic esters, for instance oils of formula R₁COOR₂ in which R₁ represents a linear or branched fatty acid residue containing from 1 to 40 carbon atoms and R₂ represents a hydrocarbon-based chain that is especially branched, containing from 1 to 40 carbon atoms provided that R₁+R₂≧10.
      The esters may be chosen especially from esters, especially fatty acid esters, for instance:
cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers, such as Lusplan DD-DA5® and Lusplan DD-DA7® sold by the company Nippon Fine Chemical and described in patent application FR 03/02809;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,
non-salified higher fatty acids such as oleic acid, linoleic acid and linolenic acid, and mixtures thereof, and
dialkyl carbonates, the two alkyl chains possibly being identical or different, such as the dicaprylyl carbonate sold under the name Cetiol CC® by Cognis,
and mixtures thereof.

The non-volatile silicone oils are chosen, for example, from non-volatile polydimethylsiloxanes (PDMSs), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, and dimethicones or phenyl trimethicones with a viscosity of less than or equal to 100 cSt, and mixtures thereof.

The non-volatile oils may be chosen from mixtures of hydrocarbon-based and silicone non-volatile oils.

### Volatile Oils

For the purposes of the present invention, the term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile hydrocarbon-based oils may be chosen from hydrocarbon-based oils containing from 8 to 16 carbon atoms, and in particular branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile fluoro oils such as nonafluoromethoxybutane or perfluoromethylcyclopentane, and mixtures thereof, may also be used.

Volatile oils that may also be used include volatile silicones, for instance volatile linear or cyclic silicone oils, especially those with a viscosity ≦8 centistokes (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made in particular of dimethicones with viscosities of 5 and 6 cSt, octamethylcyclotetrasiloxane, decamethylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

It is also possible to use a mixture of hydrocarbon-based and silicone volatile oils.

The oil(s) are preferably chosen from C₆-C₁₆ lower alkanes; linear or branched hydrocarbons of mineral or synthetic origin containing more than 16 carbon atoms; non-silicone oils of animal origin; oils of plant origin; fluoro oils; liquid fatty alcohols; liquid fatty esters; non-salified liquid fatty acids; silicone oils; or mixtures thereof, and are preferably chosen from C₆-C₁₆ lower alkanes; linear or branched hydrocarbons of mineral or synthetic origin containing more than 16 carbon atoms; liquid fatty alcohols; oils of plant origin; or mixtures thereof, and even more preferentially chosen from C₆-C₁₆ lower alkanes; linear or branched hydrocarbons, of mineral or synthetic origin, of more than 16 carbon atoms; liquid fatty alcohols; or mixtures thereof.

The oil(s) are preferably present in a content ranging from 0.1% to 20%, more preferentially in an amount ranging from 1% to 10%, and better still in an amount ranging from 1.5% to 5% by weight, relative to the total weight of the composition.

The fatty phase of the compositions also comprises one or more fatty-phase thickener(s) and especially oils.

According to the present invention, the term “fatty-phase thickener” means compounds which, by their presence, increase the viscosity of the fatty phase into which they are introduced by at least 20 cps and preferably by at least 50 cps, at 25° C. and at a shear rate of 1 s⁻¹ (the viscosity may be measured using a cone/plate viscometer, a Haake R600 rheometer or the like).

The notion of a fatty-phase thickener is analogous to the notion of a lipophilic thickener.

The fatty-phase thickener(s) used in the composition according to the invention may be mineral or organic.

The mineral fatty-phase thickeners that may be used in the composition according to the invention are preferably mineral particles consisting essentially of mineral oxides and/or hydroxides.

These particles are preferably insoluble in water at room temperature (25° C.). The term “insoluble” means a solubility of less than 0.5% by weight.

Preferably, the number-average primary size of these mineral particles ranges from 0.01 to 500 μm, it preferably ranges from 0.1 to 200 μm and even more preferentially it ranges from 1 to 100 μm.

For the purposes of the present invention, the term “primary particle size” means the maximum dimension that it is possible to measure between two diametrically opposite points on an individual particle.

The size of the mineral particles may be determined by transmission electron microscopy or by measuring the specific surface area via the BET method or by laser granulometry.

The mineral particles that may be used in accordance with the invention may be in various forms, for example in the form of spheres, needles, flakes or platelets.

In a preferred variant of the invention, the mineral fatty-phase thickener(s) are platelet-shaped particles.

The mineral fatty-phase thickener(s) that may be used in the cosmetic composition according to the invention may preferably be chosen from silicas and silicates.

The silicates of the invention may be natural or chemically modified (or synthetic).

Silicates correspond to optionally hydrated silica in which some of the silicon atoms are replaced with metal cations such as Al³⁺, B³⁺, Fe³⁺, Ga³⁺, Be²⁺, Zn²⁺, Mg²⁺, Co³⁺, Ni³⁺, Na⁺, Li⁺, Ca²⁺, Cu²⁺.

More particularly, the silicates that may be used in the context of the invention are chosen from clays of the smectite family such as montmorillonites, hectorites, bentonites, beidellites and saponites, and also of the vermiculite, stevensite and chlorite families.

These clays may be of natural or synthetic origin. Clays that are cosmetically compatible and acceptable with keratin materials are preferably used.

The silicate may be chosen from montmorillonite, bentonite, hectorite, attapulgite and sepiolite, and mixtures thereof.

Mention may thus be made of the compounds sold by the company Laporte under the name Laponite XLG and Laponite XLS.

The silicate(s) are preferably chosen from bentonites and hectorites.

The silicate(s) may be modified with a compound chosen from quaternary amines, tertiary amines, amine acetates, imidazolines, amine soaps, fatty sulfates, alkylarylsulfonates and amine oxides, and mixtures thereof.

As silicates that may be suitable for use, mention may be made of quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay; quaternium-18 hectorites such as those sold under the names Bentone Gel DOA, Bentone Gel ECO5, Bentone Gel EUG, Bentone Gel IPP, Bentone Gel ISD, Bentone Gel SS71, Bentone Gel VS8 and Bentone Gel VS38 by the company Rheox, and Simagel M and Simagel SI 345 by the company Biophil.

The silicates that may be used in the composition according to the invention may be chosen, in particular, from modified hectorites such as hectorite modified with a C₁₀-C₁₂ fatty acid ammonium chloride, especially distearyldimethylammonium chloride and stearylbenzyldimethylammonium chloride.

As explained previously, the mineral fatty-phase thickener(s) that may be used in the composition according to the invention may be silicas.

The silicas that may be used in the composition according to the invention are preferably fumed silicas.

Fumed silicas may be obtained by high-temperature hydrolysis of a volatile silicon compound in an oxyhydrogen flame, producing a finely divided silica. This process makes it possible especially to obtain hydrophilic silicas which bear a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of the said silicas, via a chemical reaction generating a reduction in the number of silanol groups. It is possible in particular to replace silanol groups with hydrophobic groups: a hydrophobic silica is then obtained.

The hydrophobic groups may be:

(a) trimethylsiloxyl groups, which are obtained especially by treating fumed silica in the presence of hexamethyldisilazane. Silicas thus treated are known as “Silica silylate” according to the CTFA (6th edition, 1995). They are sold, for example, under the references Aerosil R812® by the company Degussa and Cab-O-Sil TS-530® by the company Cabot;

(b) dimethylsilyloxyl or polydimethylsiloxane groups, which are obtained in particular by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa and Cab-O-Sil TS-610® and Cab-O-Sil TS-720® by the company Cabot.

Preferably, the fumed silicas that may be used in the composition according to the invention are hydrophilic, such as the product sold under the name Aerosil 200®.

Preferably, the mineral fatty-phase thickener(s) are chosen from organophilic clays and hydrophilic fumed silicas, and mixtures thereof.

More preferentially, the mineral fatty-phase thickeners are chosen from hectorites modified with a C₁₀-C₁₂ fatty acid ammonium chloride, especially distearyldimethylammonium chloride and stearylbenzyldimethylammonium chloride, and hydrophilic fumed silicas such as the hydrophilic silicas sold under the name Aerosil 200®.

Even more preferentially, the mineral fatty-phase thickeners are chosen from hectorites modified with a C₁₀-C₁₂ fatty acid ammonium chloride, especially hectorite modified with distearyldimethylammonium chloride, such as the product sold under the name Bentone 38VCG by the company Elementis, and the hectorite modified with stearylbenzyldimethylammonium chloride, such as the product sold under the name Bentone 27V by the company Elementis.

As explained previously, the fatty-phase thickener(s) that may be used in the composition according to the invention may also be chosen from organic fatty-phase thickeners.

The organic fatty-phase thickener(s) may be chosen especially from semicrystalline polymers, non-silicone polyamides, silicone polyamides, monoalkyl or polyalkyl esters of saccharides or of polysaccharides, N-acylamino acid amide derivatives, copolymers comprising an alkylene and/or styrene block, and elastomeric organopolysiloxanes, and mixtures thereof. These copolymers may be diblock, triblock or multi-block polymers, radial-block polymers, also known as star copolymers, or alternatively comb polymers.

Preferably, the fatty-phase thickener(s) are chosen from mineral thickeners.

More preferably, the fatty-phase thickener(s) are chosen from mineral thickeners of silicate type, more preferably from hectorites.

The fatty-phase thickeners are preferably present in a content ranging from 0.05% to 10% by weight and better still from 0.075% to 5% by weight relative to the total weight of the composition.

The composition according to the invention also comprises at least one water-insoluble mineral particulate compound, other than the fatty-phase thickener.

For the purposes of the present invention, the term “water-insoluble” refers to a compound whose solubility at spontaneous pH in water at 25° C. and at atmospheric pressure is less than 0.1%.

Preferably, the water-insoluble mineral particulate compound is a styling compound. The term “styling water-insoluble mineral particulate compound” means a water-insoluble mineral particulate compound which has a capacity for shaping the head of hair or for the durability of this shaping.

In particular, the mineral particulate compound(s) present in the composition according to the invention may have various shapes and/or sizes so as to form points of attachment between the keratin fibres onto which they are deposited.

Preferably, the mineral particulate compound(s) according to the invention are not thickeners, i.e. they do not increase, by their presence, the viscosity of the fatty phase into which they are introduced by at least 20 cps, at 25° C. and at a shear rate of 1 s⁻¹ (viscosity may be measured using a cone/plate viscometer, a Haake R600 rheometer or the like).

The mineral particulate compounds according to the invention may optionally be modified with organic groups.

Preferably, the number-mean primary size of these particles ranges from 0.01 to 500 μm, preferably ranges from 0.1 to 200 μm and even more preferentially ranges from 1 to 100 μm, better still from 1 to 50 μm.

These compounds may be in various forms, for example in the form of spheres, needles, flakes or platelets.

The water-insoluble mineral particulate compound(s) may be chosen from metal particles, oxides, mineral salts, carbides, nitrides, sulfides and hydroxides.

The term “metal particles” means particles formed from metals chosen from alkaline-earth metals, transition metals, rare-earth metals and alloys of these metals.

Preferably, the metals used are in particular boron, aluminium, copper, cadmium, selenium, silver, gold, indium, iron, platinum, nickel, molybdenum, silicon, titanium, tungsten, antimony, palladium, zinc and tin, and alloys of these metals. Among these metals, gold, silver, platinum, cadmium and selenium, and alloys of these metals, are most particularly preferred.

The particles of one or more mineral compounds may also be oxides. Mention may be made of oxides of the elements in columns 1 to 14 of the Periodic Table of the Elements. In particular, mention may be made especially of titanium oxide, zinc oxide, cerium oxide, zirconium oxide, aluminium oxide and bismuth oxychloride. Among these compounds, zinc oxide is most particularly preferred.

The particles of one or more mineral compounds may be mineral salts. Mention may be made especially of barium sulfate, calcium carbonate, calcium sulfate, calcium phosphate and magnesium hydrogen carbonate. Among these compounds, calcium carbonate is preferred.

The particles of one or more mineral compounds may be carbides, nitrides, borides, sulfides and hydroxides.

Among the particles of one or more mineral compounds belonging to the species described above, mention may also be made of alumina, silica and mineral compounds containing the same such as perlite, silicates and in particular aluminosilicates such as kaolin.

In particular, the silicas that can be used may be natural and untreated. Mention may thus be made of the silicas sold under the names Sillitin N85, Sillitin N87, Sillitin N82, Sillitin V85 and Sillitin V88 by the company Hoffmann Mineral, or Sunsil 130 by the company Sunjin Chemical, MSS-500-3 H by the company Kobo, Sunsphere H 51 by the company AGC Si-Tech, and the hollow particles of ellipsoidal amorphous silica sold by Kobo under the reference Silica Shells.

They may be fumed silicas.

The fumed silicas may be obtained by high-temperature hydrolysis of a volatile silicon compound in an oxyhydrogen flame, producing a finely divided silica. This process makes it possible especially to obtain hydrophilic silicas which contain a large number of silanol groups at their surface. It is possible to chemically modify the surface of said silica via a chemical reaction which brings about a reduction in the number of silanol groups. It is possible especially to substitute silanol groups with hydrophobic groups; a hydrophobic silica is then obtained.

The hydrophobic groups may be:

(a) trimethylsiloxyl groups, which are obtained especially by treating fumed silica in the presence of hexamethyldisilazane. Silicas thus treated are known as “silica silylate” according to the CTFA (6th edition, 1995);

(b) dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995).

In particular, among the hydrophobic silicas, mention may be made of silica aerogels.

Aerogels are ultralight porous materials which were first produced by Kristler in 1932.

They are generally synthesized by a sol-gel process in a liquid medium and then dried by extraction with a supercritical fluid. The supercritical fluid most commonly used is supercritical CO₂. This type of drying makes it possible to avoid the contraction of the pores and of the material.

Other types of drying also make it possible to obtain porous materials starting from gel, namely (i) drying by freeze drying, which consists in solidifying the gel at low temperature and in then subliming the solvent, and (ii) drying by evaporation. The materials thus obtained are referred to respectively as cryogels and xerogels. The sol-gel process and the various drying processes are described in detail in Brinker C J., and Scherer G. W., Sol-Gel Science: New York: Academic Press, 1990.

The term “hydrophobic silica” is understood to mean any silica whose surface is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

Preferably, the hydrophobic aerogel particles that may be used in the present invention advantageously have a specific surface area per unit of mass (SM) ranging from 500 to 1500 m²/g, preferably from 600 to 1200 m²/g and better still from 600 to 800 m²/g and/or have an oil-absorbing capacity measured at the Wet Point ranging from 5 to 18 ml/g of particles, preferably from 6 to 15 ml/g and better still from 8 to 12 ml/g.

The absorption capacity measured at the wet point, denoted Wp, corresponds to the amount of oil which needs to be added to 100 g of particles in order to obtain a homogeneous paste.

It is measured according to the Wet Point method or the method for determining the oil uptake of a powder according to the principle described in standard NF T 30-022. It corresponds to the amount of oil adsorbed onto the available surface of the powder and/or absorbed by the powder by measurement of the wet point, described below:

An amount m=2 g of powder is placed on a glass plate and the oil (isononyl isononanoate) is then added dropwise. After addition of 4 to 5 drops of oil to the powder, mixing is performed using a spatula, and addition of oil is continued until conglomerates of oil and powder have formed. From this point, the oil is added one drop at a time and the mixture is then triturated with the spatula. The addition of oil is stopped when a firm, smooth paste is obtained. This paste must be able to be spread over the glass plate without cracks or the formation of lumps. The volume Vs (expressed in ml) of oil used is then noted.

The oil uptake corresponds to the ratio Vs/m.

The hydrophobic silica aerogel particles used according to the present invention are preferably aerogel particles of silylated silica (INCI name: silica silylate).

The preparation of hydrophobic silica aerogel particles surface-modified by silylation is further described in document U.S. Pat. No. 7,470,725.

Use will in particular be made of aerogel particles of hydrophobic silica surface-modified with trimethylsilyl groups.

The hydrophobic aerogel particles that may be used in the present invention advantageously have a size, expressed as the mean diameter (D[0.5]), of less than 1500 μm, preferably ranging from 1 to 30 μm, preferably from 5 to 25 μm, better still from 5 to 20 μm and even better still from 5 to 15 μm.

The specific surface area per unit of mass may be determined by the nitrogen absorption method, known as the BET (Brunauer-Emmett-Teller) method, described in the Journal of the American Chemical Society, Vol. 60, page 309, February 1938 and corresponding to the international standard ISO 5794/1 (appendix D). The BET specific surface area corresponds to the total specific surface area of the particles under consideration.

The sizes of the aerogel particles according to the invention can be measured by static light scattering using a commercial particle size analyser of MasterSizer 2000 type from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles”, Chapters 9 and 10, Wiley, New York, 1957.

According to an advantageous embodiment, the hydrophobic aerogel particles used in the present invention have a specific surface area per unit of mass (SM) ranging from 600 to 800 m²/g and a size, expressed as the volume-mean diameter (D[0.5]), ranging from 5 to 20 μm and better still from 5 to 15 μm.

According to a preferred embodiment, VM-2270 will more particularly be used, the particles of which have a mean size ranging from 5 to 15 microns and a specific surface area per unit of mass ranging from 600 to 800 m²/g.

The hydrophobic aerogel particles used in the present invention may advantageously have a tapped density ρ ranging from 0.04 g/cm³ to 0.10 g/cm³ and preferably from 0.05 g/cm³ to 0.08 g/cm³.

In the context of the present invention, this density can be assessed according to the following protocol, known as the tapped density protocol:

40 g of powder are poured into a graduated measuring cylinder and then the measuring cylinder is placed on a Stay 2003 device from Stampf Volumeter. The measuring cylinder is subsequently subjected to a series of 2500 tapping actions (this operation is repeated until the difference in volume between two consecutive tests is less than 2%) and the final volume Vf of tapped powder is then measured directly on the measuring cylinder.

The tapped density is determined by the ratio: mass (m)/Vf, in this instance 40/Vf (Vf being expressed in cm³ and m in g).

According to one embodiment, the hydrophobic aerogel particles used in the present invention have a specific surface area per unit of volume SV ranging from 5 to 60 m²/cm³, preferably from 10 to 50 m²/cm³ and better still from 15 to 40 m²/cm³.

The specific surface area per unit of volume is given by the relationship:

SV=SM*ρ

where ρ is the tapped density expressed in g/cm³ and SM is the specific surface area per unit of mass expressed in m²/g, as defined above.

According to a preferred embodiment, the hydrophobic aerogel particles according to the invention have a specific surface area per unit of mass (SM) ranging from 500 to 1500 m²/g, preferably from 600 to 1200 m²/g and better still from 600 to 800 m²/g, and a size expressed as the mean diameter (D[0.5]) ranging from 1 to 30 μm and/or an oil-absorbing capacity measured at the Wet Point ranging from 5 to 18 ml/g of particles, preferably from 6 to 15 ml/g and better still from 8 to 12 ml/g.

As hydrophobic silica aerogels that may be used in the invention, an example that may be mentioned is the aerogel sold under the name VM-2260 (INCI name: silica silylate) by the company Dow Corning, the particles of which have a mean size of about 1000 microns and a specific surface area per unit of mass ranging from 600 to 800 m²/g.

Mention may also be made of the aerogels sold by Cabot under the references Aerogel TLD 201, Aerogel OGD 201 and Aerogel TLD 203, Enova Aerogel MT 1100 and Enova Aerogel MT 1200.

Use will be made more particularly of the aerogel sold under the name VM-2270 (INCI name: silica silylate), by the company Dow Corning, the particles of which have an average size ranging from 5 to 15 microns and a specific surface area per unit of mass ranging from 600 to 800 m²/g.

Preferably, the water-insoluble styling particulate mineral compounds are chosen from silicates, alumina, silica and mineral compounds containing the same such as perlite, nitrides, calcium carbonate, preferably from silica particles, especially hydrophobic silica aerogel particles, perlite, nitrides, especially boron nitride, calcium carbonate, silicates, especially aluminosilicates such as kaolin.

According to a particular embodiment, the water-insoluble particulate mineral compounds comprise at least one mineral compound with a molecular weight of less than 150 g/mol, preferably less than 100 g/mol and better still less than 80 g/mol.

The water-insoluble mineral particulate compound(s) other than the fatty-phase thickener may be present in a content ranging from 0.01% to 5%, preferably from 0.02% to 2% and better still from 0.02% to 0.5% by weight relative to the total weight of the composition.

The fatty phase of the composition may also comprise any usual liposoluble or lipodispersible additive, for instance other solid or pasty fatty substances such as waxes, fatty alcohols or fatty acids. It may also comprise compounds such as alkylene carbonates, such as propylene carbonate, which can reinforce the efficacy of certain fatty-phase thickeners such as silicates.

The amount of fatty phase may range from 0.5% to 50% by weight, preferably from 0.7% to 30% by weight and better still from 1% to 20% by weight, relative to the total weight of the composition.

As mentioned previously, the composition according to the invention comprises an aqueous phase.

The aqueous phase of the composition according to the invention comprises at least water.

The amount of water may represent at least 30% by weight, preferably at least 50% by weight and better still at least 60% by weight relative to the total weight of the composition.

The amount of water may represent from 30% to 98% by weight, preferably from 50% to 95% by weight and better still from 60% to 92% by weight relative to the total weight of the composition.

Preferably, the weight ratio of the amount of water to the amount of oil(s) in the compositions of the invention ranges from 1 to 80, better still from 5 to 70 and even better still from 10 to 60.

The aqueous phase of the composition according to the invention also comprises an aqueous-phase thickener.

According to the present invention, the term “aqueous-phase thickener” means compounds which, by their presence, increase the viscosity of the aqueous phase into which they are introduced by at least 20 cps and preferably by at least 50 cps, at 25° C. and at a shear rate of 1 s⁻¹ (the viscosity may be measured using a cone/plate viscometer, a Haake R600 rheometer or the like).

Aqueous-phase thickeners that may be mentioned include non-associative thickening polymers bearing sugar units.

For the purposes of the present invention, the term “sugar unit” means a unit derived from a carbohydrate of formula Cₙ(H₂O)ₙ₋₁ or (CH₂O)ₙ, which may be optionally modified by substitution and/or by oxidation and/or by dehydration.

The sugar units that may be included in the composition of the thickening polymers of the invention are preferably derived from the following sugars:
glucose;
galactose;
arabinose;
rhamnose;
mannose;
xylose;
fucose;
anhydrogalactose;
galacturonic acid;
glucuronic acid;
mannuronic acid;
galactose sulfate;
anhydrogalactose sulfate and
fructose.

Thickening polymers of the invention that may especially be mentioned include native gums such as:

a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (or tragacanth) (polymer of galacturonic acid, galactose, fucose, xylose and arabinose);
   b) gums derived from algae, including:
agar (polymer derived from galactose and anhydrogalactose);
alginates (polymers of mannuronic acid and glucuronic acid);
carrageenans and furcellerans (polymers of galactose sulfate and anhydrogalactose sulfate);
   c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);
   d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
   e) plant extracts, including:
cellulose (glucose polymer);
starch (glucose polymer) and
inulin.

These polymers may be physically or chemically modified. A physical treatment that may especially be mentioned is the temperature.

Chemical treatments that may be mentioned include esterification, etherification, amidation or oxidation reactions. These treatments can lead to polymers that may especially be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly)hydroxyalkyl groups.

Among the C₁-C₆ (poly)hydroxyalkyl groups that may be mentioned, for example, are hydroxymethyl, hydroxyethyl, hydroxypropyl and hydroxybutyl groups.

These guar gums are well known in the prior art and can be prepared, for example, by reacting the corresponding alkene oxides, for instance propylene oxides, with the guar gum so as to obtain a guar gum modified with hydroxypropyl groups.

The degree of hydroxyalkylation preferably ranges from 0.4 to 1.2, and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified especially by one or more of the following reactions: pregelatinization, oxidation, crosslinking, esterification, etherification, amidation, heat treatments.

Distarch phosphates or compounds rich in distarch phosphate will preferentially be used, for instance the products sold under the references Prejel VA-70-T AGGL (gelatinized hydroxypropyl cassava distarch phosphate), Prejel TK1 (gelatinized cassava distarch phosphate) and Prejel 200 (gelatinized acetyl cassava distarch phosphate) by the company Avebe, or Structure Zea from National Starch (gelatinized corn distarch phosphate).

According to the invention, amphoteric starches may also be used, these amphoteric starches comprising one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The starch molecules may be derived from any plant source of starch, especially such as corn, potato, oat, rice, tapioca, sorghum, barley or wheat. It is also possible to use the starch hydrolysates mentioned above. The starch is preferably derived from potato.

The non-associative thickening polymers of the invention may be cellulose-based polymers not comprising a C₁₀-C₃₀ fatty chain in their structure.

According to the invention, the term “cellulose-based” polymer means any polysaccharide compound bearing in its structure sequences of glucose residues linked via β-1,4 bonds; besides unsubstituted celluloses, the cellulose derivatives may be anionic, cationic, amphoteric or nonionic.

Thus, the cellulose-based polymers of the invention may be chosen from unsubstituted celluloses, including those in a microcrystalline form, and cellulose ethers.

Among these cellulose-based polymers, cellulose ethers, cellulose esters and cellulose ester ethers are distinguished.

Among the cellulose esters are mineral esters of cellulose (cellulose nitrates, sulfates, phosphates, etc.), organic cellulose esters (cellulose monoacetates, triacetates, am idopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers without a C₁₀-C₃₀ fatty chain, i.e. which are “non-associative”, mention may be made of (C₁-C₄)alkylcelluloses, such as methylcelluloses and ethylcelluloses (for example, Ethocel standard 100 Premium from

Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl-(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly)carboxy(C₁-C₄)alkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and the sodium salts thereof.

Among the cationic cellulose ethers without a fatty chain, mention may be made of cationic cellulose derivatives such as cellulose copolymers or cellulose derivatives grafted with a water-soluble quaternary ammonium monomer, and described in particular in patent U.S. Pat. No. 4,131,576, such as (poly)hydroxy(C₁-C₄)alkyl celluloses, for instance hydroxymethyl-, hydroxyethyl- or hydroxypropylcelluloses grafted in particular with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

Among the nonassociative thickening polymers not bearing sugar units that may be used, mention may be made of crosslinked acrylic or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or mixtures thereof.

A first family of nonassociative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by the company Noveon or the products sold under the names Synthalen M and Synthalen K by the company 3 VSA.

The nonassociative thickening polymers may also be crosslinked (meth)acrylic acid copolymers, such as the polymer sold under the name Aqua SF1 by the company Noveon.

The nonassociative thickening polymers may be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of document EP 503 853, and reference may be made to said document as regards these polymers.

The composition may similarly comprise, as nonassociative thickening polymers, ammonium acrylate homopolymers or copolymers of ammonium acrylate and of acrylamide.

Among the ammonium acrylate homopolymers that may be mentioned is the product sold under the name Microsap PAS 5193 by the company Hoechst. Among the copolymers of ammonium acrylate and of acrylamide that may be mentioned is the product sold under the name Bozepol C Nouveau or the product PAS 5193 sold by the company Hoechst. Reference may be made especially to documents FR 2 416 723, U.S. Pat. No. 2,798,053 and U.S. Pat. No. 2,923,692 as regards the description and preparation of such compounds.

Cationic thickening polymers of acrylic type may also be used.

Among the aqueous-phase thickening polymers, mention may also be made of the associative polymers that are well known to a person skilled in the art and especially of nonionic, anionic, cationic or amphoteric nature.

It is recalled that “associative polymers” are polymers that are capable, in an aqueous medium, of reversibly associating with each other or with other molecules. Their chemical structure more particularly comprises at least one hydrophilic region and at least one hydrophobic region.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type that may be mentioned are:
(a) those comprising at least one hydrophilic unit and at least one fatty-chain allyl ether unit, more particularly those whose hydrophilic unit is formed by an ethylenic unsaturated anionic monomer, more particularly by a vinylcarboxylic acid and most particularly by an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate and methylenebisacrylamide.

Among the latter polymers, those most particularly preferred are crosslinked terpolymers of methacrylic acid, of ethyl acrylate and of polyethylene glycol (10 EO) stearyl alcohol ether (Steareth 10), in particular those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10).
(b) those comprising i) at least one hydrophilic unit of unsaturated olefinic carboxylic acid type, and ii) at least one hydrophobic unit of the type such as a (C₁₀-C₃₀) alkyl ester of an unsaturated carboxylic acid.

(C₁₀-C₃₀) alkyl esters of unsaturated carboxylic acids that are useful in the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to patents U.S. Pat. No. 3,915,921 and U.S. Pat. No. 4,509,949.

Among anionic associative polymers of this type that will be used more particularly are those consisting of from 95% to 60% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among the said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP.
(c) maleic anhydride/C₃₀-C₃₈ α-olefin/alkyl maleate terpolymers, such as the product (maleic anhydride/C₃₀-C₃₈ α-olefin/isopropyl maleate copolymers) sold under the name Performa V 1608® by the company Newphase Technologies.
(d) acrylic terpolymers comprising:

i) about 20% to 70% by weight of an α,β-monoethylenically unsaturated carboxylic acid [A],

ii) about 20% to 80% by weight of a non-surfactant monomer containing α,β-monoethylenic unsaturation other than [A],

iii) about 0.5% to 60% by weight of a nonionic monourethane which is the product of reaction of a monohydric surfactant with a monoisocyanate containing monoethylenic unsaturation,

such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(e) copolymers comprising among their monomers a carboxylic acid containing α,β-monoethylenic unsaturation and an ester of a carboxylic acid containing α,β-monoethylenic unsaturation and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas.
(f) amphiphilic polymers comprising at least one ethylenically unsaturated monomer bearing a sulfonic group, in free or partially or totally neutralized form and comprising at least one hydrophobic part. These polymers may be crosslinked or non-crosslinked. They are preferably crosslinked.

The ethylenically unsaturated monomers bearing a sulfonic group are especially chosen from vinylsulfonic acid, styrenesulfonic acid, (meth)acrylamido(C₁-C₂₂)alkylsulfonic acids, N-(C₁-C₂₂)alkyl(meth)acrylamido(C₁-C₂₂)alkylsulfonic acids such as undecylacrylamidomethanesulfonic acid, and also partially or totally neutralized forms thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used.

2-Acrylamido-2-methylpropanesulfonic acid (AMPS), and also partially or totally neutralized forms thereof, will more particularly be used.

The polymers of this family may be chosen especially from random amphiphilic AMPS polymers modified by reaction with a C₆^{-C}₂₂ n-monoalkylamine or di-n-alkylamine, and such as those described in patent application WO 00/31154 (which is an integral part of the content of the description). These polymers may also contain other ethylenically unsaturated hydrophilic monomers selected, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid and maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not comprising a fatty chain, such as (meth)acrylic acids, ⊖-substituted alkyl derivatives thereof or esters thereof obtained with monoalcohols or mono- or polyalkylene glycols, (meth)acrylamides, vinylpyrrolidone, maleic anhydride, itaconic acid and maleic acid, or mixtures of these compounds.

These copolymers are described especially in patent application EP-A-750 899, patent U.S. Pat. No. 5,089,578 and in the following Yotaro Morishima publications:
“Self-assembling amphiphilic polyelectrolytes and their nanostructures—Chinese Journal of Polymer Science, Vol. 18, No. 40, (2000), 323-336”;
“Micelle formation of random copolymers of sodium 2-(acrylamido)-2-methylpropanesulfonate and a nonionic surfactant macromonomer in water as studied by fluorescence and dynamic light scattering—Macromolecules, Vol. 33, No. 10, (2000), 3694-3704”;
“Solution properties of micelle networks formed by nonionic moieties covalently bound to a polyelectrolyte: salt effects on rheological behavior—Langmuir, Vol. 16, No. 12, (2000) 5324-5332”;
“Stimuli responsive amphiphilic copolymers of sodium 2-(acrylamido)-2-methylpropanesulfonate and associative macromonomers'Polym. Preprint, Div. Polym. Chem., 40(2), (1999), 220-221”.

Among these polymers, mention may be made of:
crosslinked or non-crosslinked, neutralized or non-neutralized copolymers, comprising from 15% to 60% by weight of AMPS units and from 40% to 85% by weight of (C₈-C₁₆)alkyl(meth)acrylamide or (C₈-C₁₆)alkyl(meth)acrylate units relative to the polymer, such as those described in patent application EP-A750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in patent U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Among the cationic associative polymers that may be mentioned are:

(I) cationic associative polyurethanes;
(II) the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.
   (III) quaternized (poly)hydroxyethylcelluloses modified with groups comprising at least one fatty chain, such as alkyl, arylalkyl or alkylaryl groups comprising at least 8 carbon atoms, or mixtures thereof. The alkyl radicals borne by the above quaternized celluloses or hydroxyethylcelluloses preferably comprise from 8 to 30 carbon atoms. The aryl radicals preferably denote phenyl, benzyl, naphthyl or anthryl groups. Examples of quaternized alkylhydroxyethylcelluloses containing C₈-C₃₀ fatty chains that may be indicated include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Aqualon, and the products Crodacel QM®, Crodacel QL® (C₁₂ alkyl) and Crodacel QS® (C₁₈ alkyl) sold by the company Croda and the product Softcat SL 100® sold by the company Aqualon.
   (IV) cationic polyvinyllactam polymers.

Such polymers are described, for example, in patent application WO-00/68282.

As cationic poly(vinyllactam) polymers according to the invention, vinylpyrrolidone/dimethylaminopropylmethacrylamide/dodecyldimethylmethacrylamidop ropylammonium tosylate terpolymers, vinylpyrrolidone/dimethylaminopropyl-methacrylamide/cocoyldimethylmethacrylamidopropylammonium tosylate terpolymers, vinylpyrrolidone/dimethylaminopropylmethacrylamide/lauryldimethylmethacrylamido-propylammonium tosylate or chloride terpolymers are used in particular.

The amphoteric associative polymers are preferably chosen from those comprising at least one non-cyclic cationic unit. Even more particularly, the ones that are preferred are those prepared from or comprising 1 mol % to 20 mol %, preferably 1.5 mol % to 15 mol % and even more particularly 1.5 mol % to 6 mol % of fatty-chain monomer relative to the total number of moles of monomers.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:
(a) copolymers of vinylpyrrolidone and of fatty-chain hydrophobic monomers, of which examples that may be mentioned include:
   the products Antaron V216® or Ganex V216® (vinylpyrrolidone/hexadecene copolymer) sold by the company I.S.P.,
   the products Antaron V220® or Ganex V220® (vinylpyrrolidone/eicosene copolymer) sold by the company I.S.P.,
(b) copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name Antil 208®,
(c) copolymers of hydrophilic methacrylates or acrylates and of hydrophobic monomers comprising at least one fatty chain, for instance the polyethylene glycol methacrylate/lauryl methacrylate copolymer,
(d) polyurethane polyethers comprising in their chain both hydrophilic blocks usually of polyoxyethylenated nature and hydrophobic blocks, which may be aliphatic sequences alone and/or cycloaliphatic and/or aromatic sequences,
(e) polymers with an aminoplast ether backbone containing at least one fatty chain, such as the Pure Thix® compounds sold by the company Sud-Chemie,
(f) celluloses or derivatives thereof, modified with groups comprising at least one fatty chain, such as alkyl, arylalkyl or alkylaryl groups or mixtures thereof in which the alkyl groups are of C₈, and in particular:
nonionic alkylhydroxyethylcelluloses such as the products Natrosol Plus Grade 330 CS and Polysurf 67 (C₁₆ alkyl) sold by the company Aqualon;
nonionic nonoxynylhydroxyethylcelluloses such as the product Amercell HM-1500 sold by the company Amerchol;
nonionic alkylcelluloses such as the product Bermocoll EHM 100 sold by the company Berol Nobel;
(g) associative guar derivatives, for instance hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be envisaged. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane bond between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂₋₁₄ alkyl chain, and the product Elfacos T212® containing a C₁₈ alkyl chain, from Akzo.

The product DW 1206B® from Röhm & Haas containing a C₂₀ alkyl chain and a urethane bond, sold at a solids content of 20% in water, may also be used.

Use may also be made of solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Röhm & Haas may also be used.

The polyurethane polyethers that may be used according to the invention are in particular those described in the article by G. Fonnum, J. Bakke and Fk. Hansen—Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold in particular by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the aqueous-phase thickener(s) are chosen from polymers not comprising any sugar units.

Preferably, the aqueous-phase thickener(s) are chosen from anionic thickening polymers.

More preferentially, the aqueous-phase thickener(s) are chosen from associative or non-associative polymers bearing acrylic or methacrylic units.

The aqueous-phase thickener or thickeners is or are preferably present in a content ranging from 0.1% to 20%, more preferentially in an amount ranging from 0.2% to 15% and better still in an amount ranging from 0.5% to 10% by weight, relative to the total weight of the composition.

The aqueous phase may comprise at least one hydrophilic organic solvent, for instance substantially linear or branched lower monoalcohols containing from 1 to 8 carbon atoms, such as ethanol, propanol, butanol, isopropanol or isobutanol; polyols, such as propylene glycol, isoprene glycol, butylene glycol, glycerol, sorbitol, polyethylene glycols and derivatives thereof; and mixtures thereof.

Preferably, the composition according to the invention does not comprise any surfactant. When it does comprise the same, the composition according to the invention comprises less than 2% of surfactant.

The amount of aqueous phase may range from 50% to 99.5% by weight, preferably from 60% to 95% by weight and better still from 70% to 90% by weight, relative to the total weight of the composition.

The composition according to the invention may comprise active agents conventionally used in the field of cosmetics, other than those described previously, and chosen from fixing polymers, preferably anionic or non-ionic fixing polymers, silicones, direct dyes, in particular cationic or natural direct dyes, or oxidation dyes, organic or mineral pigments, UV-screening agents, resins, fragrances, peptizers, vitamins, amino acids, preserving agents, long-lasting hair shaping agents, especially thiolated organic reducing agents, non-thiolated organic reducing agents, alkaline agents, etc.

Needless to say, a person skilled in the art will take care to select the optional additional compounds and/or the amount thereof such that the advantageous properties of the compositions used according to the invention are not, or are not substantially, adversely affected by the envisaged addition.

According to a preferred embodiment, the composition comprises:
a fatty phase comprising:

a) at least one oil;

b) at least one fatty-phase thickener chosen from silicates;

c) at least one water-insoluble mineral particulate compound different from the fatty-phase thickener b),
an aqueous phase comprising at least one aqueous-phase thickener chosen from associative or non-associative anionic thickening polymers bearing acrylic or methacrylic units.

Preferably, the composition according to the invention does not comprise any superabsorbent polymer, namely a polymer that is capable in its dry form of spontaneously absorbing at least 20 times its own weight of aqueous fluid, in particular of water and especially distilled water.

Preferably, the composition is in the form of a gel, namely a thickened aqueous solution, which comprises oily inclusions, such as oily volutes. More preferably, the composition is in the form of a transparent gel with oily inclusions such as oily volutes. More preferably, the composition is entirely in gel form, the two phases being thickened.

Preferably, the compositions have a viscosity of greater than or equal to 0.1 Pa·s and better still ranging from 0.1 Pa·s to 500 Pa·s and even better still from 0.5 Pa·s to 300 Pa·s and even more preferably from 1 Pa·s to 200 Pa·s at a temperature of 25° C. and at a shear rate of 1 s⁻¹ (measurable, for example, with a Haake RS600 rheometer).

The composition according to the invention may be obtained by mixing the two phases using a static mixer.

In particular, to make the composition according to the invention, the ingredients of the fatty phase are mixed together, on the one hand, and the ingredients of the aqueous phase are mixed together, on the other hand. Each phase is introduced separately into the static mixer, namely a tube inside which is a three-dimensional structure promoting the appearance of turbulence during the passage of a fluid. The phases are mixed by a static device, i.e. a device that is not driven by a rotary system, thus avoiding dispersion of the fatty phase in the aqueous phase, especially in the form of globules. A mixture in which the two phases are visually distinct is obtained.

A subject of the invention is also a composition according to the invention made using a static mixer.

The composition according to the invention may especially be used in leave-in or rinse-out application to the hair.

A subject of the invention is also a cosmetic hair treatment process, which consists in applying to the hair an effective amount of a composition as has just been described, followed by optionally rinsing it out after an optional leave-in time, in the presence or absence of heat.

The example that follows is given as an illustration of the present invention. In this example, all the amounts are indicated as weight percentages of active material (AM) relative to the total weight of the composition.

| | Name | A |
|---|---|---|
| 1 | Mineral oil | 1.76 |
| 2 | Mica (and) iron oxides (and) titanium | 0.02 |
| | dioxide | |
| 3 | Disteardimonium hectorite ⁽¹⁾ | 0.12 |
| 4 | Boron nitride | 0.04 |
| 5 | Propylene Carbonate | 0.04 |
| 6 | Fragrance | 0.01 |
| 7 | Tocopherol | 0.01 |
| 8 | CARBOMER ⁽²⁾ | 1.27 |
| 9 | Caprylyl glycol | 0.39 |
| 10 | PEG-40 hydrogenated castor oil | 1.18 |
| 11 | Triethanolamine | 1.86 |
| 12 | Polysorbate 20 | 0.10 |
| 13 | Disodium EDTA | 0.08 |
| 14 | Fragrance | 0.39 |
| 15 | Preserving agent | qs |
| 16 | Water | qs 100 |

⁽¹⁾ sold under the reference Bentone 38 V CG by the company Elementis
⁽²⁾ sold under the reference Synthalen K by the company 3V

The composition, which is a styling gel with a care valency, is prepared.

Using a static mixer, the oil phase comprising ingredients 1 to 6 is mixed with the gel phase comprising the other ingredients of the composition. The composition obtained is in the form of a marbled, translucent gel containing aesthetic whitish volutes.

The gel obtained is sparingly tacky and easy to spread in the hands and on the hair.

According to a first use, it is applied to wet hair before brushing. It gives the hair texture and volume.

According to a second use, it is applied to dry hair, as a finishing. It is applied after straightening or curling the hair using a straightening or curling iron, and affords hairstyle control and hold.

In the two examples of use, the styling obtained has long-lasting flexible hold with no “cardboard” effect. In addition, the hair has a soft, cosmetic feel.

1. Cosmetic hair treatment process comprising the step of applying on the hair a composition comprising:
a fatty phase comprising:
a) at least one oil;
b) at least one fatty-phase thickener;
c) at least one water-insoluble mineral particulate compound, other than the fatty-phase thickener b);
an aqueous phase comprising at least one aqueous-phase thickening polymer, the two phases being visually distinct.

2. Process according to the preceding claim, characterized in that the oil(s) are chosen from C₆-C₁₆ lower alkanes; linear or branched hydrocarbons of mineral or synthetic origin containing more than 16 carbon atoms; non-silicone oils of animal origin; oils of plant origin; fluoro oils; liquid fatty alcohols; liquid fatty esters; non-salified liquid fatty acids; silicone oils; or mixtures thereof; and are preferably chosen from C₆-C₁₆ lower alkanes; linear or branched hydrocarbons of mineral or synthetic origin containing more than 16 carbon atoms; liquid fatty alcohols; oils of plant origin; or mixtures thereof; and even more preferentially chosen from C₆-C₁₆ lower alkanes; linear or branched hydrocarbons, of mineral or synthetic origin, of more than 16 carbon atoms; liquid fatty alcohols; or mixtures thereof.

3. Process composition according to either of the preceding claims, characterized in that the oil(s) are present in an amount ranging from 0.1% to 20%, more preferentially in an amount ranging from 1% to 10% and better still in an amount ranging from 1.5% to 5% by weight, relative to the total weight of the composition.

4. Process according to any one of the preceding claims, characterized in that the fatty-phase thickener is chosen from mineral fatty-phase thickeners and organic fatty-phase thickeners.

5. Process according to the preceding claim, characterized in that the mineral fatty-phase thickeners are chosen from silicates and silicas, preferably from silicates.

6. Process according to claim 4, characterized in that the organic fatty-phase thickeners are chosen from semicrystalline polymers, non-silicone polyamides, silicone polyamides, monoalkyl or polyalkyl esters of saccharides or of polysaccharides, N-acylamino acid amide derivatives, copolymers comprising one or more alkylene and/or styrene blocks, and elastomeric organopolysiloxanes, and mixtures thereof.

7. Process according to any one of the preceding claims, characterized in that the fatty-phase thickener or thickeners is or are present in a content ranging from 0.05% to 10% by weight relative to the total weight of the composition and preferably from 0.075% to 5% by weight relative to the total weight of the composition.

8. Process according to any one of the preceding claims, characterized in that the water-insoluble mineral particulate compound is chosen from metal particles, oxides, mineral salts, carbides, nitrides, sulfides and hydroxides, especially from silicates, alumina, silica and mineral compounds containing the same such as perlite, nitrides, calcium carbonate, preferably from silica particles, especially hydrophobic silica aerogel particles, perlite, nitrides, especially boron nitride, silicates, especially aluminosilicates such as kaolin.

9. Process according to any one of the preceding claims, characterized in that the water-insoluble mineral particulate compound(s) are present in a content ranging from 0.01% to 5%, preferably from 0.02% to 2% and better still from 0.02% to 0.5% by weight, relative to the total weight of the composition.

10. Process according to any one of the preceding claims, characterized in that the amount of fatty phase ranges from 0.5% to 50% by weight, preferably from 0.7% to 30% by weight and better still from 1% to 20% by weight, relative to the total weight of the composition.

11. Process according to any one of the preceding claims, characterized in that the aqueous-phase thickener is chosen from non-associative thickening polymers bearing sugar units, non-associative thickening polymers not bearing sugar units and associative thickening polymers.

12. Process according to any one of the preceding claims, characterized in that the aqueous-phase thickener is chosen from anionic thickening polymers.

13. Process according to any one of the preceding claims, characterized in that the aqueous-phase thickener is chosen from associative or non-associative thickening polymers bearing acrylic or methacrylic units.

14. Process according to any one of the preceding claims, characterized in that the aqueous-phase thickener or thickeners is or are present in an amount ranging from 0.1% to 20%, more preferentially in an amount ranging from 0.2% to 15% and better still in an amount ranging from 0.5% to 10% by weight, relative to the total weight of the composition.

15. Process according to any one of the preceding claims, characterized in that the amount of aqueous phase ranges from 50% to 99.5% by weight, preferably from 60% to 95% by weight and better still from 70% to 90% by weight, relative to the total weight of the composition.

16. Process according to any one of the preceding claims, characterized in that the composition comprises less than 2% of surfactant and preferably does not comprise any surfactant.

17. Process according to any one of the preceding claims, characterized in that the composition has a viscosity of greater than or equal to 0.1 Pa·s, better still ranging from 0.1 Pa·s to 500 Pa·s, even better still from 0.5 Pa·s to 300 Pa·s and more preferably from 1 Pa·s to 200 Pa·s at a temperature of 25° C. and at a shear rate of 1 s⁻¹.

18. Process according to any one of the preceding claims, characterized in that the composition is in gel form with oily insertions such as oily volutes.

19. Process according to any one of the preceding claims, characterized in that the composition is obtained by mixing the two phases using a static mixer.

20. Process according to any one of the preceding claims, characterized in that it further comprises the step of rinsing the composition out after an optional leave-in time, in the presence or absence of heat.

21. Use of the composition according to any one of claims 1 to 19 for caring for the hair and/or for shaping the hair.
